(19) United States
(12) Patent Application Publication
(10) Pub. No.: US 2008/0003603 A1
(43) Pub. Date: Jan. 3, 2008

(54) HUMAN MELANOMA MUTATION

(76) Inventors: Joseph A. Holden, Salt Lake City, UT (US); Carlynn Willmore-Payne, Riverton, UT (US); Lester J. Layfield, Salt Lake City, UT (US)

Correspondence Address: BRINKS HOFER GILSON & LIONE;UTAH OFFICE, 405 South Main Street, Suite 800, SALT LAKE CITY, UT, 84111-3400, US

(21) Appl. No.: 11/741,543

(22) Filed: Apr. 27, 2007

Related U.S. Application Data
(60) Provisional application No. 60/745,836, filed on Apr. 27, 2006.
(60) Provisional application No. 60/804,232, filed on Jun. 8, 2006.

(51) Int. Cl. C07H 21/04; C12Q 1/68

(52) U.S. Cl. 435/6; 536/23.1; 536/24.3; 536/24.33

(57) ABSTRACT

The present invention relates to the field of melanoma diagnosis and therapy. In particular, the present invention relates to c-kit activating mutations present in melanoma, and their use for melanoma evaluation and diagnosis, melanoma prognosis, melanoma progression monitoring, and prescribing therapeutic strategies such as treatment with imatinib or other kinase inhibitors.

### CROSS REFERENCE TO RELATED APPLICATION

This application claims the benefit under 35 U.S.C. § 119(e) of U.S. Provisional Patent Application No. 60/745,836, filed Apr. 27, 2006, and titled “Human Malignant Melanoma Mutation,” and of U.S. Provisional Patent Application No. 60/804,232, filed Jun. 8, 2006, and titled “Human Melanoma Mutation,” which are incorporated, in their entirety, by this reference.

### FIELD OF THE INVENTION

The present invention relates to the field of melanoma diagnosis and therapy. In particular, the present invention relates to c-kit activating mutations present in melanoma, and their use for melanoma evaluation and diagnosis, melanoma prognosis, melanoma progression monitoring, and prescribing therapeutic strategies such as treatment with imatinib or other kinase inhibitors.

### BACKGROUND OF THE INVENTION

Protein kinases are members of a large family of proteins which play important roles in cellular signaling. Several types of human malignancies contain specific mutations in genes coding for protein kinases that may represent primary oncogenic events. These mutations result in the production of an activated protein kinase which is no longer sensitive to normal inhibitory signals. Constant signaling by such mutationally activated proteins stimulates cell proliferation, activates anti-apoptotic pathways, and drives oncogenesis. Several new types of anticancer drugs are available which are inhibitory against the activated protein kinases. Because these activated protein kinases are unique to the tumor cell, the drugs which inhibit them are far less toxic than standard anticancer drugs as they target only the malignant cell. This type of targeted molecular therapy is highlighted by the successful treatment of gastrointestinal stromal tumors (GISTs), chronic myelogenous leukemia (CML), and adenocarcinomas of the lung with tyrosine kinase inhibitors such as imatinib and gefitinib. Such successes suggest that other hard to treat malignancies may be characterized by specific mutations which result in activated proteins, sensitive to targeted anticancer drugs.

The human c-kit protein is a transmembrane receptor tyrosine kinase. It has structural similarity with platelet-derived growth factor receptor, fms-like tyrosine kinase-3 receptor, and macrophage colony-stimulating factor receptor. Together these molecules represent a subclass III of membrane-bound tyrosine kinase receptors. Binding of its ligand, stem cell factor, leads to c-kit homodimerization and activation of its intrinsic tyrosine kinase activity. Activation of the tyrosine kinase leads to autophosphorylation at tyrosines located at positions 568, 570, 703, 721, and 936 (1-3). These autophosphorylated tyrosines physically associate with cytoplasmic proteins containing src homology domains such as the STAT proteins (4). This association leads to an intra-cellular signaling cascade resulting in cell proliferation. Normally, c-kit is expressed in hematopoietic precursors, mast cells, melanocytes, germ cells, and the interstitial cells of Cajal. Gain-of-function mutations in the human c-kit protein have been observed in a variety of human tumors, including germ cell tumors (5) mast cell tumors (6), and gastrointestinal stromal tumors (GISTs) (7). These mutations result in c-kit activation independent of ligand, and this process is thought to be the driving force that results in neoplastic growth (8). One of the recent successes of targeted molecular therapy has been the finding that imatinib mesylate (STI-571, Gleevec) inhibits the activated c-kit receptor in GISTs (9). This new drug is a phenylaminopyrimidine derivative. It is a competitive inhibitor of the c-kit tyrosine kinase. Patients with GISTs who previously had few therapeutic options can be treated with imatinib mesylate, and many show partial responses and stabilization of disease (10-12). This underscores the need to provide an accurate identification and diagnosis of other neoplasms in which c-kit activating mutations are present.

Malignant melanoma is a difficult to treat malignancy. Immune based therapy has received the widest attention. However, treatment with immune system modifiers such as interleukin-2 and interferon alpha have not been shown to provide convincing survival benefit and suffer from severe toxicity. Melanoma vaccines have also met with limited success. Chemotherapeutic drugs such a dacarbazine and newer combinations of drugs such as temozolomide and thalidomide are active against the disease but response rates are only around 20%. Thus, progress in treating melanoma has been slow and significant advances may require new insights into melanoma biology.

Melanomas have also been reported to overexpress c-kit. However, no c-kit mutations, which probably are a prerequisite for imatinib response, have been previously described in melanoma.

Identification and detection of activating mutations the c-kit gene that are associated with melanoma prognosis or response to treatment would be highly beneficial. Such genetic variations, typically in the form of single nucleotide polymorphisms (SNPs), arising from a deletion or insertion of a nucleotide or substitution of one nucleotide for another at the polymorphic site, could be used as “markers” to classify tumors and predict response to therapy.

### SUMMARY OF THE INVENTION

The present invention relates to the discovery of c-kit activating mutations associated with melanoma that can potentially be used for prognosis and development of drugs for clinical treatment of melanoma.

High resolution amplicon melting analysis has been previously used to characterize the c-kit activating mutations in a series of gastrointestinal stromal tumors (GISTs). In accordance with the present invention, high resolution melting analysis was used to search for c-kit activating mutations in order to characterize the genotype of a series of malignant melanomas. It was also discovered that two cases of melanoma which showed strong expression of the c-kit protein both contained an identical activating mutation in the c-kit gene at exon 11, resulting in a C→T base-pair change in nucleotide 69495 of NCBI U63834, resulting in a mutation characterized as c-kit L576P. Alterations in exon 11 are the most common genetic abnormality present in GISTs (13-18). Exon 11 codes for the cytoplasmic juxtamembrane domain of the protein. This domain exerts a negative regulatory effect on the c-kit protein. When this domain is disrupted, spontaneous c-kit activation occurs (19).

The SNPs disclosed herein can be used as targets for the design of diagnostic reagents and the development of therapeutic agents. Mutations in the c-kit genes may be utilized, for example, as targets to identify protein kinase inhibitors specific for the mutation that are capable of deactivating the c-kit gene and thereby treating malignant melanoma. In addition, such mutations may be used for diagnostic purposes for rapid characterization of c-kit mutations in tumors. The SNPs of the present invention are useful for providing clinically important information for treatment of melanoma, and for screening and selecting therapeutic agents. Methods, assays, kits, and reagents for detecting the presence of these polymorphisms and their encoded products are provided.

Based on the identification of c-kit mutations associated with melanoma, the present invention also provides methods of detecting these variants as well as the design and preparation of detection reagents needed to accomplish this task. The invention specifically provides activating c-kit mutations in genetic sequences involved in melanoma, variant proteins encoded by nucleic acid molecules containing such SNPs, methods of detecting these SNPs in a test sample and methods of identifying individuals who are more or less likely to respond to a treatment.

In particular embodiments, the present invention also relates to a specific mutation in the c-kit gene resulting in a C→T base-pair change in nucleotide 69495 of NCBI U63834 (in exon 11) of the c-kit gene, resulting in a mutation characterized as c-kit L576P.

One aspect of the present invention relates to an isolated nucleic acid molecule comprising a nucleotide sequence in which at least one nucleotide is a SNP resulting in the mutation L576P. In an alternative embodiment, a nucleic acid of the invention is an amplified polynucleotide, which is produced by amplification of a SNP-containing nucleic acid template. In another embodiment, the invention provides for a variant protein which is encoded by a nucleic acid molecule containing a SNP disclosed herein.

In yet another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest. In an alternative embodiment, a protein detection reagent is used to detect a variant protein which is encoded by a nucleic acid molecule containing a SNP disclosed herein. A preferred embodiment of a protein detection reagent is an antibody or an antigen-reactive antibody fragment.

Also provided in the invention are kits comprising SNP detection reagents, and methods for detecting the SNPs disclosed herein by employing detection reagents. In a specific embodiment, the present invention provides for a method of identifying an individual having a melanoma that might be susceptible to a specific treatment, such as treatment with imatinib or other kinase inhibitors, by detecting the presence or absence of a SNP allele disclosed herein.

Many other uses and advantages of the present invention will be apparent to those skilled in the art upon review of the detailed description of the preferred embodiments herein. Solely for clarity of discussion, the invention is described in the sections below by way of non-limiting examples.

### DETAILED DESCRIPTION OF THE INVENTION

The present invention provides SNPs that were previously known in the art, but were not previously known to be associated with melanoma. Accordingly, the present invention provides novel compositions and methods of detecting and using the SNPs disclosed herein, and also provides novel methods of using the known, but previously unassociated, SNPs in methods relating to melanoma. The SNPs disclosed herein are useful for evaluating melanomas and identifying those which might be susceptible to specific treatments, such as treatment with imatinib or other kinase inhibitors. Furthermore, such SNPs and their encoded products are useful targets for the screening and development of therapeutic agents.

The present invention provides SNPs associated with melanoma, nucleic acid molecules containing SNPs, methods and reagents for the detection of the SNPs disclosed herein, uses of these SNPs for the development of detection reagents, and assays or kits that utilize such reagents. As used herein, terms such as “SNP,” “polymorphism,” “mutation,” “mutant,” “variation,” and “variant” are equivalent and interchangeable.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, thymine (uridine), cytosine, or guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position, reference is generally made to the protein-encoding strand, only for the purpose of convenience.

Isolated Nucleic Acid Molecules

The present invention provides isolated nucleic acid molecules that contain SNPs resulting in L576P substitution. In some embodiments of the present invention, the SNP is characterized by a T→C base-pair change in exon 11 of c-kit. Isolated nucleic acid molecules containing one or more SNPs may be interchangeably referred to throughout the present text as “SNP-containing nucleic acid molecules.” Isolated nucleic acid molecules may optionally encode a full-length variant protein or fragment thereof. The isolated nucleic acid molecules of the present invention also include probes and primers (which are described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” generally is one that contains a SNP of the present invention or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated.” Nucleic acid molecules present in non-human transgenic animals, which do not naturally occur in the animal, are also considered “isolated.” For example, recombinant DNA molecules contained in a vector are considered “isolated.” Further examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. Preferably the flanking sequence is up to about 500, 300, 100, 60, 50, 30, 25, 20, 15, 10, 8, or 4 nucleotides (or any other length in-between) on either side of a SNP position, or as long as the full-length gene or entire protein-coding sequence (or any portion thereof such as an exon), especially if the SNP-containing nucleic acid molecule is to be used to produce a protein or protein fragment.

For full-length genes and entire protein-coding sequences, a SNP flanking sequence can be, for example, up to about 5 KB, 4 KB, 3 KB, 2 KB, 1 KB on either side of the SNP. Furthermore, in such instances, the isolated nucleic acid molecule comprises exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs disclosed herein are also encompassed by the present invention, and such fragments may be used, for example, to express any part of a protein, such as a particular functional domain or an antigenic epitope.

Thus, the present invention also encompasses fragments of the nucleic acid sequences encompassing the SNPs disclosed herein, contiguous nucleotide sequence at least about 8 or more nucleotides, alternatively at least about 12 or more nucleotides, and alternatively at least about 16 or more nucleotides. Further, a fragment could comprise at least about 15, 18, 20, 22, 25, 30, 40, 50, 60, 100, 250 or 500 (or any other number in-between) nucleotides in length. The length of the fragment will be based on its intended use. For example, the fragment can encode epitope-bearing regions of a variant peptide or regions of a variant peptide that differ from the normal/wild-type protein, or can be useful as a polynucleotide probe or primer. Such fragments can be isolated using all or part of the nucleotide sequences provided in SEQ ID NO:1 for the synthesis of a polynucleotide probe. A labeled probe can then be used, for example, to screen a cDNA library, genomic DNA library, or mRNA to isolate nucleic acid corresponding to the coding region. Further, primers can be used in amplification reactions, such as for purposes of assaying one or more SNPs sites or for cloning specific regions of a gene.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In some embodiments of the invention, an amplified polynucleotide is at least about 30 nucleotides in length. In another embodiment of the invention, an amplified polynucleotide is at least about 32, 40, 45, 50, or 60 nucleotides in length. In yet another embodiment of the invention, an amplified polynucleotide is at least about 100, 200, or 300 nucleotides in length. While the total length of an amplified polynucleotide of the invention can be as long as an exon, an intron or the entire gene where the SNP of interest resides, an amplified product is typically no greater than about 1,000 nucleotides in length (although certain amplification methods may generate amplified products greater than 1000 nucleotides in length). Generally, an amplified polynucleotide is not greater than about 600 nucleotides in length. It is understood that irrespective of the length of an amplified polynucleotide, a SNP of interest may be located anywhere along its sequence.

In a specific embodiment of the invention, the amplified product is at least about 201 nucleotides in length, comprises one of the transcript-based context sequences or the genomic-based context sequences shown in SEQ ID NO:1. Such a product may have additional sequences on its 5′ end or 3′ end or both. In another embodiment, the amplified product is about 101 nucleotides in length, and it contains a SNP disclosed herein. Preferably, the SNP is located at the middle of the amplified product (e.g., at position 101 in an amplified product that is 201 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present invention provides isolated nucleic acid molecules that comprise, consist of, or consist essentially of one or more polynucleotide sequences that contain one or more SNPs disclosed herein, complements thereof, and SNP-containing fragments thereof.

Accordingly, the present invention provides nucleic acid molecules that consist of any of the nucleotide sequences encompassing the L576P mutation. A nucleic acid molecule consists of a nucleotide sequence when the nucleotide sequence is the complete nucleotide sequence of the nucleic acid molecule.

A nucleic acid molecule consists essentially of a nucleotide sequence when such a nucleotide sequence is present with only a few additional nucleotide residues in the final nucleic acid molecule.

The present invention further provides nucleic acid molecules that comprise any of the nucleotide sequences encoding the L576P mutation. Nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated is provided below, and such techniques are well known to those of ordinary skill in the art (Sambrook and Russell, 2000, Molecular Cloning: A Laboratory Manual, Cold Spring Harbor Press, NY).

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition,” Trends Biotechnol. 1997 June; 15(6):224-9, and Hyrup et al., “Peptide nucleic acids (PNA): synthesis, properties and potential applications,” Bioorg. Med. Chem. 1996 January; 4(1):5-23). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs. Furthermore, kits/systems (such as beads, arrays, etc.) that include these analogs are also encompassed by the present invention. For example, PNA oligomers that are based on the polymorphic sequences of the present invention are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994), Petersen et al., Bioorganic & Medicinal Chemistry Letters, 6:793-796 (1996), Kumar et al., Organic Letters 3(9):1269-1272 (2001), WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

SNP Detection Reagents

In a specific aspect of the present invention, the SNPs of the invention can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a probe that hybridizes to a target nucleic acid containing one or more of the SNPs. In a preferred embodiment, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding to the context sequences. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

In one preferred embodiment of the invention, a SNP detection reagent is an isolated or synthetic DNA or RNA polynucleotide probe or primer or PNA oligomer, or a combination of DNA, RNA and/or PNA, that hybridizes to a segment of a target nucleic acid molecule containing a SNP identified in SEQ ID NO:1. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer typically is a substantially purified oligonucleotide or PNA oligomer. Such oligonucleotide typically comprises a region of complementary nucleotide sequence that hybridizes under stringent conditions to at least about 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

It will be apparent to one of skill in the art that such primers and probes are directly useful as reagents for genotyping the SNPs of the present invention, and can be incorporated into any kit/system format.

In order to produce a probe or primer specific for a target SNP-containing sequence, the gene/transcript and/or context sequence surrounding the SNP of interest is typically examined using a computer algorithm which starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides,” “allele-specific probes,” or “allele-specific primers.” The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5× standard saline phosphate EDTA (SSPE), 0.5% NaDodSO4 (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ end or the 3′ end of the probe or primer. In a specific preferred embodiment which is particularly suitable for use in an oligonucleotide ligation assay (U.S. Pat. No. 4,988,617), most of the nucleotide of the probe aligns with the SNP position in the target sequence.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position. Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity (Gibbs, 1989, Nucleic Acid Res. 17:2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan assay, described below.

In a specific embodiment of the invention, a primer of the invention contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment of the invention, a SNP detection reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the invention, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl. 4:357-362; Tyagi et al., 1996, Nature Biotechnology 14: 303-308; Nazarenko et al., 1997, Nucl. Acids Res. 25:2516-2521; U.S. Pat. Nos. 5,866,336 and 6,117,635).

The detection reagents of the invention may also contain other labels, including but not limited to, biotin for streptavidin binding, hapten for antibody binding, and oligonucleotide for binding to another complementary oligonucleotide such as pairs of zip codes.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the 3′-most end of the primer extension product, and in which the ddNTP corresponds to a SNP disclosed herein, is a composition that is encompassed by the present invention). Thus, reagents that bind to a nucleic acid molecule in a region adjacent to a SNP site, even though the bound sequences do not necessarily include the SNP site itself, are also encompassed by the present invention.

SNP Detection Kits and Systems

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems,” as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting one or more SNPs, including the SNPs shown in SEQ ID NO:1.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 15-25 nucleotides in length. In further embodiments, a nucleic acid array can comprise any number of probes, in which at least one probe is capable of detecting one or more SNPs disclosed in SEQ ID NO:1, and/or at least one probe comprises a fragment of one of the sequences selected from the group consisting of those disclosed in SEQ ID NO:1, the Sequence Listing, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a novel SNP allele disclosed in SEQ ID NO:1. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA) from skin, biopsies, or tissue specimens. Methods of preparing nucleic acids are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM 6700, and Roche Molecular Systems'COBAS AmpliPrep System.

The present invention thus provides kits for detecting a single nucleotide polymorphisms (SNP) in a nucleic acid. In some embodiments, the present invention is directed to kits for detecting a single nucleotide polymorphisms (SNP) in a nucleic acid, comprising the polynucleotide described above that are capable of detecting the presence or absence of the c-kit L576P activating mutation. Such kits may also contain a buffer, and an enzyme, such as a polymerase enzyme for primer extension. In other embodiments, the present invention is directed to kits for identifying a drug susceptibility-conferring mutation, comprising: a suitable container, and at least one of the following: a wild-type c-kit polynucleotide; at least one c-kit polynucleotide comprising a drug resistance-conferring mutation; or a primer that identifies the resistance-conferring mutation.

Uses of Nucleic Acid Molecules

The nucleic acid molecules of the present invention have a variety of uses, especially in the evaluation and treatment of melanoma. For example, the nucleic acid molecules are useful as hybridization probes, such as for genotyping SNPs in messenger RNA, transcript, cDNA, genomic DNA, amplified DNA or other nucleic acid molecules, and for isolating full-length cDNA and genomic clones encoding the variant peptides disclosed in SEQ ID NO:2 as well as their orthologs.

A probe can hybridize to any nucleotide sequence along the entire length of a nucleic acid molecule provided in SEQ ID NO:1. In some embodiments, a probe of the present invention hybridizes to a region of a target sequence that encompasses a SNP position indicated in SEQ ID NO:1. In other embodiments, a probe hybridizes to a SNP-containing target sequence in a sequence-specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. These uses are relevant for diagnosis of disorders involving an increase or decrease in gene expression relative to normal levels. In vitro techniques for detection of mRNA include, for example, Northern blot hybridizations and in situ hybridizations. In vitro techniques for detecting DNA include Southern blot hybridizations and in situ hybridizations (Sambrook and Russell, 2000, Molecular Cloning: A Laboratory Manual, Cold Spring Harbor Press, Cold Spring Harbor, N.Y.).

Probes can be used as part of a diagnostic test kit for identifying cells or tissues in which a variant protein is expressed, such as by measuring the level of a variant protein-encoding nucleic acid (e.g., mRNA) in a sample of cells from a subject or determining if a polynucleotide contains a SNP of interest.

The nucleic acid molecules of the invention are also useful as primers to amplify any given region of a nucleic acid molecule, particularly a region containing a SNP.

The nucleic acid molecules of the invention are also useful in assays for drug screening to identify compounds that, for example, modulate nucleic acid expression.

SNP Genotyping Methods

The process of determining which specific nucleotide (i.e., allele) is present at each of one or more SNP positions, such as a SNP position in a nucleic acid molecule, is referred to as SNP genotyping. The present invention provides methods of SNP genotyping, such as for use in diagnosing or evaluating melanoma or related pathologies, or determining responsiveness to a form of treatment.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput,” Pharmacogenomics J. 2003; 3(2):77-96; Kwok et al., “Detection of single nucleotide polymorphisms,” Curr Issues Mol. Biol. 2003 April; 5(2):43-60; Shi, “Technologies for individual genotyping: detection of genetic polymorphisms in drug targets and melanoma genes,” Am. J. Pharmacogenomics. 2002; 2(3):197-205; and Kwok, “Methods for genotyping single nucleotide polymorphisms,” Annu. Rev. Genomics Hum. Genet. 2001; 2:235-58. Exemplary techniques for high-throughput SNP genotyping are described in Marnellos, “High-throughput SNP analysis for genetic association studies,” Curr. Opin. Drug Discov. Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and S1 protection or chemical cleavage methods. In one embodiment, SNP genotyping is performed using the TaqMan assay, which is also known as the 5′ nuclease assay (U.S. Pat. Nos. 5,210,015 and 5,538,848).

TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for melanoma and related pathologies, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another method for genotyping the SNPs of the present invention is the use of two oligonucleotide probes in an OLA (see, e.g., U.S. Pat. No. 4,988,617). The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6054564 describe OLA strategies for performing SNP detection; WO 97/31256 and WO 00/56927 describe OLA strategies for performing SNP detection using universal arrays, wherein a zip code sequence can be introduced into one of the hybridization probes, and the resulting product, or amplified product, hybridized to a universal zip code array; U.S. application US01/17329 (and 09/584,905) describes OLA (or LDR) followed by PCR, wherein zip codes are incorporated into OLA probes, and amplified PCR products are determined by electrophoretic or universal zip code array readout; U.S. application 60/427,818, 60/445,636, and 60/445,494 describe SNPlex methods and software for multiplexed SNP detection using OLA followed by PCR, wherein zip codes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays. The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol. Biol. 2003; 212:241-62; Jurinke et al., “The use of MassARRAY technology for high throughput genotyping”, Adv Biochem Eng Biotechnol. 2002; 77:57-74; and Jurinke et al., “Automated genotyping using the DNA MassArray technology”, Methods Mol. Biol. 2002; 187:179-92.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

Other methods that can be used to genotype the SNPs of the present invention include single-strand conformational polymorphism (SSCP), denaturing gradient gel electrophoresis (DGGE) (Myers et al., Nature 313:495 (1985)). Sequence-specific ribozymes (U.S. Pat. No. 5,498,531) can also be used to score SNPs based on the development or loss of a ribozyme cleavage site.

SNP genotyping and detection can include the steps of, for example, collecting a biological sample from a human subject, isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping and detection is useful for numerous practical applications. Examples of such applications include, but are not limited to melanoma diagnosis, melanoma prognosis, melanoma progression monitoring, determining and prescribing therapeutic strategies based on an individual's genotype (“pharmacogenomics”), or developing therapeutic agents based on SNP genotypes associated with a melanoma or likelihood of responding to a drug, as well as stratifying a patient population for a clinical trial of a treatment regimen.

Melanoma Evaluation

Information on association/correlation between genotypes and melanoma-related phenotypes can be exploited in several ways. In the present case, the presence of the c-kit L576P mutation indicates that a melanoma tumor is more susceptible to treatment by specific drugs, such as imatinib.

As used herein, the terms “diagnose”, “diagnosis”, and “diagnostics” include, but are not limited to any of the following: detection or evaluation of melanoma that an individual may presently have, determining a particular type or subclass of melanoma in an individual known to have melanoma, confirming or reinforcing a previously made diagnosis of melanoma, pharmacogenomic evaluation of an individual to determine which therapeutic strategy that individual is most likely to positively respond to or to predict whether a patient is likely to respond to a particular treatment, and evaluating the future prognosis of an individual having melanoma. Such diagnostic uses are based on the SNPs individually or in combination with other SNPs associated with melanoma.

The present invention includes methods of determining the genotype of a melanoma tissue, for purposes of evaluating appropriate therapeutic treatment. In some embodiments, the present invention relates to a method for determining the presence of an activating c-kit mutation in melanoma, comprising detecting a polymorphic variant of SEQ ID NO:1 encoding c-kit L576P, or a complement thereof. In some embodiments, the present invention is directed to a method of detecting a single nucleotide polymorphism (SNP) in a nucleic acid molecule, comprising: contacting a test sample with a reagent that specifically hybridizes to the SNP in a nucleotide sequence encoding c-kit L576P, or a complement thereof, under stringent hybridization conditions, and detecting the formation of a hybridized duplex. In other embodiments, the present invention is directed to a method of determining whether an individual will be responsive to cancer treatment with a kinase inhibitor, comprising providing a nucleic acid sample of the individual, and detecting in the nucleic acid sample the presence or absence of a polymorphic variant encoding the c-kit L576P activating mutation. In more particular embodiments, the cancer is melanoma. In other particular embodiments, the kinase inhibitor is imatinib. In still other particular embodiments, the present invention includes the step of detecting the presence or absence of the polymorphic variant encoding the c-kit L576P activating mutation comprises detecting hybridization of an oligonucleotide probe complementary to the nucleic acid encoding the c-kit L576P activating mutation, or a complement thereof. In yet other embodiments, the present invention includes the step of detecting the presence or absence of the polymorphic variant encoding the c-kit L576P activating mutation comprises detecting hybridization of an oligonucleotide primer complementary to the nucleic acid encoding the c-kit L576P activating mutation, or a complement thereof. In yet other embodiments, the present invention includes the step of detecting the polymorphic variant encoding the c-kit L576P activating mutation includes subjecting said primer to polymerization conditions, such that polymerization of the primer is indicative of the presence in the c-kit polynucleotide of the c-kit L576P activating mutation. In more particular embodiment of the invention, the individual in need of screening for susceptibility to treatment by a kinase inhibitor has been determined to be resistant to a cancer drug other than a kinase inhibitor.

Pharmacogenomics and Therapeutics/Drug Development

The present invention provides methods for assessing the pharmacogenomics of a subject harboring particular SNP alleles or haplotypes to a particular therapeutic agent or pharmaceutical compound, or to a class of such compounds.

Pharmacogenomic uses of the SNPs of the present invention provide several significant advantages for patient care, particularly in treating melanoma. Pharmacogenomic characterization of an individual, based on an individual's SNP genotype, can identify those individuals unlikely to respond to treatment with a particular medication and thereby allows physicians to avoid prescribing the ineffective medication to those individuals. On the other hand, SNP genotyping of an individual may enable physicians to select the appropriate medication and dosage regimen that will be most effective based on an individual's SNP genotype. This information increases a physician's confidence in prescribing medications and motivates patients to comply with their drug regimens. Thus, pharmacogenomics based on the SNPs disclosed herein has the potential to both save lives and reduce healthcare costs substantially.

The SNPs of the present invention are also useful for improving many different aspects of the drug development process. For example, individuals can be selected for clinical trials based on their SNP genotype. Individuals with SNP genotypes that indicate that they are most likely to respond to the drug can be included in the trials and those individuals whose SNP genotypes indicate that they are less likely to or would not respond to the drug, or suffer adverse reactions, can be eliminated from the clinical trials. This not only improves the safety of clinical trials, but also will enhance the chances that the trial will demonstrate statistically significant efficacy. Furthermore, the SNPs of the present invention may explain why certain previously developed drugs performed poorly in clinical trials and may help identify a subset of the population that would benefit from a drug that had previously performed poorly in clinical trials, thereby “rescuing” previously developed drugs, and enabling the drug to be made available to a particular melanoma patient population that can benefit from it.

### EXAMPLES

Sources of Tissue and Immunohistochemistry. Paraffin blocks from 100 cases diagnosed as malignant melanoma were retrieved from the surgical pathology files at the University of Utah Hospital. Of these 100 cases, 84 were metastatic melanomas, 12 were primary cutaneous melanomas, and 4 were in situ melanomas. For the 12 primary cutaneous melanomas, the average Breslow depth was 5.5 mm (range 1-14 mm). No primary mucosal melanomas were evaluated. The cases spanned the years from 1995 to 2004 and were chosen by sequential dates in the pathology archives. Care was taken to ensure that each case represented a lesion from a separate patient. All cases and accompanying immunohistochemical studies were reviewed to confirm the diagnosis of malignant melanoma. Only cases that were estimated to contain at least 50% tumor were analyzed, and most cases were estimated to contain between 70-90% tumor. For c-kit analysis all 100 cases were immunohistochemically stained for CD 117. Only cases showing some CD 117 staining were subsequently analyzed by molecular studies for activating mutations in the c-kit gene. The use of human tissue for this analysis was approved by the Institutional Review Board (IRB #11903) at the University of Utah.

Antibodies against c-kit (CD 117) were used at a dilution of 1:400 and were obtained from DAKO Corporation (Carpinteria, Calif.) as described previously (20). Immunohistochemical staining was performed with the Nexes Instrument from Ventana Inc. (Tucson, Ariz.) in accord with the manufacturer's instructions. The chromogen was diaminobenzidine. Mast cells, which are CD 117 positive, served as internal controls. No antigen retrieval techniques were used.

Design of Primers. Primers specific for exons 9, 11, 13, and 17 for the human c-kit gene (National Center for Biotechnology Information identification number U63834) were designed with the use of Primer Designer Software (Scientific and Education Software, Durham, N.C.). The sequences for the primers are shown in Table 1. Primers for exon 11 yield an amplicon size of 219 base pairs (bp), primers for exon 9, 235 bp; primers for exon 13, 227 bp; and primers for exon 17, 170 bp. The size of the amplicons was confirmed by subjecting the PCR products to nondenaturing polyacryl-amide gel electrophoresis on 8.0% polyacrylamide gels in a Mini-Protean II gel electrophoresis apparatus (BioRad, Hercules, Calif.) with a running buffer of a 50-mmol/L.

TABLE 1
Primers for c-kit Exons* Primer Sequence

| | | | | |
|---|---|---|---|---|
| Exon 11 | Forward | 5′-CTCTCCAGAGTGCTCTAATGAC-3′ | (SEQ ID NO: 3) | +TL,39 |
| | Reverse | 5′-AGCCCCTGTTTCATACTGACC-3′ | (SEQ ID NO: 4) | |
| Exon 9 | Forward | 5′-GATGCTCTGCTTCTGTACTG-3′ | (SEQ ID NO: 5) | |
| | Reverse | 5′-GCCTAAACATCCCCTTAAATTGG-3′ | (SEQ ID NO: 6) | |
| Exon 13 | Forward | 5′-CGGCCATGACTGTCGCTGTAA-3′ | (SEQ ID NO: 7) | |
| | Reverse | 5′-CTCCAATGGTGCAGGCTCCAA-3′ | (SEQ ID NO: 8) | |
| Exon 17 | Forward | 5′-TCTCCTCCAACCTAATAGTG-3′ | (SEQ ID NO: 9) | |
| | Reverse | 5′-GGACTGTCAAGCAGAGAAT-3′ | (SEQ ID NO: 10) | |

DNA isolation from paraffin embedded tissue. An appropriate paraffin block containing tumor tissue was selected for analysis after reviewing the hematoxylin and eosin (H and E) stained slides. An area of tumor on the H and E slide was identified on a corresponding unstained slide and circled with an indelible fine tip pen. DNA was isolated from material scraped from the unstained slide in procedures previously described (20). To obtain DNA from in-situ melanomas, or from the junctional component of invasive tumors, laser capture microdissection with a Pixcell IIe Laser Capture Microdissection Instrument and Cap-Sure Macro LCM Caps (Arcturus, Mountain View, Calif.) was used with a laser diameter of 7.5 micrometers, laser power of 45-65 mWatts, and pulse duration of 3.5-5.0 seconds. Approximately 200-300 laser pulses per specimen were used to capture the region of interest. The captured cells were incubated in 25 ul of proteinase K digestion buffer and DNA isolated as described previously (20).

Polymerase Chain Reaction. Polymerase chain reaction was performed with the Light Cycler (Roche Diagnostics, Indianapolis Ind.) as described previously (20) except that the annealing step for the BRAF exons was at 60° C. All reactions contained dUTP in place of dTTP so that incubation of the reactions with uracil N-glycosylase prior to PCR prevents “carry over” contamination. Genomic DNA was used as a control. All samples were run in triplicate.

High resolution amplicon melting. After PCR, the samples were momentarily heated to 95° C. and then cooled to 40° C. High resolution amplicon melting analysis of the PCR products was performed with the use of the HR-1 instrument (Idaho Technology, Salt Lake City, Utah) as described (20). Mixing experiments with mutant (V600E) and wild type DNA indicate that at least 50% of the total DNA isolated should be derived from the mutant in order to easily detect the mutation by melting curve analysis (data not shown).

DNA sequencing. DNA sequencing and analysis was performed as described previously (20). All cases, whether demonstrating a normal, abnormal, or indeterminate melting curve were subjected to direct DNA sequence analysis.

Expression of c-kit in Malignant Melanoma. One hundred cases of malignant melanoma were immunohistochemically stained for c-kit. No antigen retrieval techniques were used. Eleven cases contained in-situ melanoma or the junctional component of an invasive lesion and in both the in situ melanomas as well as in the junctional component of the invasive lesions, there was strong positive immunohistochemical staining for c-kit. In all of the cases of invasive lesions, the strong c-kit expression seen in the junctional component was absent in the invasive component. These findings agree with results reported by others (21, 22) suggesting that most melanomas lose c-kit expression during tumor progression. Of the 100 cases of melanoma, only 29 (29%) showed any evidence of c-kit expression and the staining ranged from weak and focal to strong and diffuse.

Expression of c-kit in Malignant Melanoma. Immunohistochemical staining for c-kit was performed as described above. Strong and diffuse c-kit staining was observed in the junctional component of an invasive cutaneous melanoma. A case of metastatic melanoma to the skin also shows strong and diffuse c-kit staining. Mutation analysis indicated that the junctional component of the invasive lesion is negative for c-kit mutation but the metastatic lesion contains a L576P mutation in c-kit exon 11.

High Resolution Amplicon Melting Analysis in c-kit Expressing Malignant Melanoma. Although most cases of malignant melanoma did not appear to overexpress c-kit, some did, and in several cases the intensity of the immunostain was similar to that observed in c-kit mutation positive gastrointestinal stromal tumors (GISTs) (20). In order to determine if any c-kit expressing melanomas contained a c-kit activating mutation, all 29 c-kit positive cases, without regard to the intensity or distribution of the c-kit immunostain, were subjected to high resolution amplicon melting analysis for c-kit exons 9, 11, 13, and 17. Surprisingly, it was found that two cases showed abnormal melting curves for c-kit exon 11. These two cases were both metastatic lesions and both showed diffuse and strong intensity c-kit immunostaining. Both cases underwent direct DNA sequencing and the abnormal melting curves for each were found to be the result of T to a C base pair change in exon 11 resulting in L576P. No normal allele was present in either case suggesting these mutations were either homozygous or hemizygous. Isolation of DNA from normal tissue from these two cases did not show the mutation suggesting this represents a somatic mutation in the tumor. Subsequent independent DNA isolation and melting analysis from each case confirmed the presence of the mutation in the tumor. No other c-kit exon activating mutations were observed in these two positive cases or in any of the other c-kit IHC positive cases. The L576P mutation has been previously reported in a GIST (18).

The frequency of c-kit mutations in malignant melanoma suggested by these results (2%) must be a minimum estimate. C-kit negative cases were not evaluated for possible c-kit mutations. Lack of c-kit expression does not rule out an early mutation in the c-kit gene with subsequent down-regulation of gene expression.

As part of this study, 100 cases of malignant melanoma were evaluated for c-kit expression by IHC. Activating mutations in c-kit had not been previously reported in melanoma. The c-kit protein is normally expressed in the interstial cells of Cajal, germ cells, mast cells, and melanocytes (23). As might be expected, activation of c-kit by mutation has been found in human tumors arising from cells of these lineages such as GISTs (24), seminomas (25) and mast cell tumors (26). What is surprising, is that although c-kit is expressed in melanocytes, c-kit activating mutations have not been found in melanoma. Perhaps this is because the mechanism of tumorigenesis in melanoma involving c-kit is fundamentally different than that involved in other neoplasms derived from cells with c-kit lineages.

In melanoma, it appears that c-kit is highly expressed in the in situ and in the junctional component of invasive lesions, but expression is lost once the melanoma becomes invasive and metastatic (27, 21, 22). Laboratory studies suggest that this is because the loss of c-kit expression allows the melanoma cells to escape from kit ligand (stem cell factor) induced apoptosis (28). Immunohistochemical staining data support this, as all in situ melanomas, as well as the junctional component of invasive melanomas, showed strong positive c-kit expression. In the cases of invasive melanomas with both junctional and invasive components, none of the invasive lesions showed c-kit expression in spite of the strong c-kit expression in their corresponding junctional component. In fact, the majority of the melanoma cases were negative for c-kit expression. C-kit immunostaining was observed in only 29 cases (29%). Some of the cases showed focal weak staining and were included as positive although the significance and importance of weak and focal c-kit staining is not clear.

Overexpression of c-kit has been associated with activating mutations in the c-kit gene (4) and so cases of melanoma were evaluated showing positive c-kit expression for c-kit activating mutations. Although all in situ lesions or the junctional component of invasive lesions showed strong c-kit expression, none contained a c-kit mutation. However, three cases of metastatic melanoma showed strongly positive and diffuse c-kit expression and the intensity of the immunostain was similar to that which were previously observed in c-kit mutation positive GISTs (20). Two of these three cases contained a c-kit exon 11 activating mutation, L576P, as determined by high resolution melting analysis and direct DNA sequencing. No mutation was found in c-kit exons 9, 13, and 17. These were the only two c-kit IHC positive cases found to contain a c-kit activating mutation. Both of these cases were metastatic lesions (primary tumors were not evaluated). The absence of the mutation in normal tissue from these two c-kit mutation positive cases suggests this is a somatic mutation in the tumor and the ability to repeatedly demonstrate the mutation in subsequent independent DNA isolations suggests it is not a technical artifact. Both c-kit mutation positive tumors were from different patients and both were histologically and immunohistochemically malignant melanoma (strongly S1100 and melan A positive). Neither case showed BRAF mutations. These results suggest that the model whereby tumor progression in melanoma is dependent on the loss of c-kit from the melanoma cell (28) or the acquisition of activating BRAF mutations (29, 30, 31), may only be partially correct. From these results, it is postulated that there may be a third, less common pathway, to melanoma tumor progression which involves not the loss of c-kit or the activation of BRAF, but rather c-kit activation through somatic mutation. Although this pathway may not be common, it is estimated that it may only occur in only 2 of 100 patients (2%), and its importance is underscored by the availability of imatinib to target activated c-kit proteins. Such patients could be readily identified by c-kit immunohistochemical staining and subsequent melting analysis. Interestingly, the same L576P mutation was recently reported in 1 of 39 cases of malignant melanoma (37). Future clinical trials to determine the efficacy of imatinib in patients with c-kit mutation positive melanoma might be considered.

The presence of c-kit activating mutations in malignant melanoma suggests a possible role for the use of specific tyrosine kinase inhibitors in the treatment of this disease. The ability to easily detect c-kit mutations by high resolution melting analysis should help guide therapy.

### REFERENCES

1. Price D J, Rivnay B, Fu Y, et al. Direct association of Csk homologous kinase (CHK) with the diphosphorylated site Tyr568/570 of the activated c-kit in megakaryocytes. J Biol Chem. 1997; 272:5915-5920.
2. Thommes K, Lennartsson J, Carlberg M, et al. Identification of Tyr-703 and Tyr-936 as the primary association sites for Grb2 and Grb7 in the c-kit/stem cell factor receptor. Biochem J. 1999; 341(pt 1):211-216.
3. Mol C D, Lim K B, Sridhar V, et al. Structure of a c-kit product complex reveals the basis for kinase transactivation. J Biol Chem. 2003; 278:31461-31464.
4. Brizzi M F, Dentelli P, Rosso A, et al. STAT protein recruitment and activation in c-kit deletion mutants. J Biol Chem. 1999; 274:16965-16972.
5. Sakuma Y, Sakurai S, Oguni S, et al. Alterations of the c-kit gene in testicular germ cell tumors. Cancer Sci. 2003; 94:486-491.
6. Nagata H, Worobec A S, Oh C K, et al. Identification of a point mutation in the catalytic domain of the protooncogene c-kit in peripheral blood mononuclear cells of patients who have mastocytosis with an associated hematologic disorder. Proc Natl Acad Sci USA. 1995; 92:10560-10564.
7. Hirota S, Isozaki K, Moriyama Y, et al. Gain-of-function mutations of c-kit in human gastrointestinal stromal tumors. Science. 1998; 279:577-580.
8. Corless C L, McGreevey L, Haley A, et al. kit mutations are common in incidental gastrointestinal stromal tumors one centimeter or less in size. Am J Pathol. 2002; 160:1567-1572.
9. Joensuu H, Roberts P J, Sarlomo-Rikala M, et al. Effect of the tyrosine kinase inhibitor STI571 in a patient with a metastatic gastrointestinal stromal tumor. N Engl J Med. 2001; 344:1052-1056.
10. Verweij J, van Oosterom A, Blay J Y, et al. Imatinib mesylate (STI-571, Glivec, Gleevec) is an active agent for gastrointestinal stromal tumours, but does not yield responses in other soft-tissue sarcomas that are unselected for a molecular target: results from an EORTC Soft Tissue and Bone Sarcoma Group phase II study. Eur J Cancer. 2003; 39:2006-2011.
11. Demetri G D, von Mehren M, Blanke C D, et al. Efficacy and safety of imatinib mesylate in advanced gastrointestinal stromal tumors. N Engl J Med. 2002; 347:472-480.
12. Croom K F, Perry C M. Imatinib mesylate: in the treatment of gastrointestinal stromal tumours. Drugs. 2003; 63:513-524.
13. Singer S, Rubin B P, Lux M L, et al. Prognostic value of kit mutation type, mitotic activity, and histologic subtype in gastrointestinal stromal tumors. J Clin Oncol. 2002; 18:3898-3905.
14. Heinrich M C, Corless C L, Demetri G D, et al. Kinase mutations and imatinib response in patients with metastatic gastrointestinal stromal tumor. J Clin Oncol. 2003; 21:4342-4349.
15. Antonescu C R, Sommer G, Sarran L, et al. Association of kit exon 9 mutations with nongastric primary site and aggressive behavior: kit mutation analysis and clinical correlates of 120 gastrointestinal stromal tumors. Clin Cancer Res. 2003; 9:3329-3337.
16. Rubin B P, Singer S, Tsao C, et al. kit activation is a ubiquitous feature of gastrointestinal stromal tumors. Cancer Res. 2001; 61:8118-8121.
17. Taniguchi M, Nishida T, Hirota S, et al. Effect of c-kit mutation on prognosis of gastrointestinal stromal tumors. Cancer Res. 1999; 59:4297-4300.
18. Wardelmann E, Neidt I, Bierhoff E, et al. c-kit mutations in gastrointestinal stromal tumors occur preferentially in the spindle rather than in the epithelioid cell variant. Mod Pathol. 2002; 15:125-136.
19. Ma Y, Cunningham M E, Wang X, et al. Inhibition of spontaneous receptor phosphorylation by residues in a putative alpha-helix in the kit intracellular juxtamembrane region. J Biol Chem. 1999; 274:13399-13402.
20. Willmore C, Holden J A, Zhou L, Tripp S, Wittwer C T, Layfield L J. Detection of c-kit-activating mutations in gastrointestinal stromal tumors by high resolution amplicon melting analysis. Am J Clin Pathol. 2004; 122:206-16.
21. Montone K T, van Bell P, Elenitsas R, Elder D E. Proto-oncogene c-kit expression in malignant melanoma: protein loss with tumor progression. Mod Pathol. 1997; 10:939-44.
22. Natali P G, Nicotra M R, Winkler A B, Cavaliere R, Bigotti A, Ullrich A. Progression of human cutaneous melanoma is associated with loss of expression of c-kit proto-oncogene receptor. Int J Cancer 1992; 52:197-201.
23. Gibson P C, Cooper K, CD117 (KIT): a diverse protein with selective applications in surgical pathology. Adv Anat Pathol 2002; 9:65-9.
24. Corless C L, Fletcher J A, Heinrich M C. Biology of gastrointestinal stromal tumors, J Clin Oncol 2004; 22:3813-25.
25. Kemmer K, Corless C L, Fletcher J A, McGreevey L, Haley A, Griffith D, et al. Kit mutations are common in testicular seminomas. Am J Pathol 2004; 164:305-13.
26. Longley B J, Metcalfe D D, Tharp M, Wang X, Tyrrell L, Lu S-Z, et al. Activating and dominant inactivating c-KIT catalytic domain mutations in distinct clinical forms of human mastocytosis. Proc Natl Acad Sci USA 1999; 96:1609-14.
27. Kovach-Guerriere P M, Hunt E L, Patterson J W, Glembocki D J, English III J C, Wick M R. Primary melanoma of the skin and cutaneous melanomatous metastases: comparative histologic features and immunophenotype. Am J Clin Pathol 2004; 122:70-7.
28. Leslie M C, Bar-Eli M. Regulation of gene expression in melanoma: new approaches for treatment. J Cell Biochem 2005; 94:25-38.
29. Dibb N J, Dilworth S M, Mol C D. Switching on kinases: oncogenic activation of BRAF and the PDGFR family, Nat Rev Cancer 2004; 4:718-27.
30. Hingorani S R, Jacobetz M A, Robertson G P, Herlyn M, Tuveson D A. Suppression of BRAFV^{V599E} in human melanoma abrogates transformation. Cancer Res 2003; 63:5198-202.
31. Sumimoto H, Miyagishi M, Miyoshi H, Yamagat S, Shimizu A, Taira K, et al. Inhibition of growth and invasive ability of melanoma by inactivation of mutated BRAF with lentivirus-mediated RNA interference. Oncogene 2004; 23:6031-9.

What is claimed is:

1. An isolated polynucleotide comprising a polymorphic variant of SEQ ID NO:1 encoding c-kit L576P, or a complement thereof.

2. The isolated polynucleotide of claim 1, comprising at least 8 contiguous nucleotides.

3. The isolated polynucleotide of claim 1, comprising at least 15 contiguous nucleotides.

4. The isolated polynucleotide of claim 1, comprising at least 20 contiguous nucleotides.

5. An amplified polynucleotide comprising a polymorphic variant of SEQ ID NO:1 encoding c-kit L576P, or a complement thereof, wherein the amplified polynucleotide is between about 16 and about 1,000 nucleotides in length.

6. An isolated polynucleotide that is capable of specifically hybridizing to a nucleic acid molecule comprising a polymorphic variant of SEQ ID NO:1 encoding c-kit L576P, or a complement thereof.

7. The polynucleotide of claim 6, wherein the polynucleotide is 8-70 nucleotides in length.

8. The polynucleotide of claim 6, wherein the polynucleotide is an allele-specific probe.

9. The polynucleotide of claim 6, wherein the polynucleotide is an allele-specific primer.

10. The polynucleotide of claim 6, wherein the polynucleotide comprises a nucleotide sequence capable of specifically hybridizing to the nucleotide sequence encoding c-kit L576P, or a complement thereof.

11. A method for determining the presence of an activating c-kit mutation in melanoma, comprising detecting a polymorphic variant of SEQ ID NO:1 encoding c-kit L576P, or a complement thereof.

12. A method of detecting a single nucleotide polymorphism (SNP) in a nucleic acid molecule, comprising:
contacting a test sample with a reagent that specifically hybridizes to the SNP in a nucleotide sequence encoding c-kit L576P, or a complement thereof, under stringent hybridization conditions, and
detecting the formation of a hybridized duplex.

13. A method of determining whether an individual will be responsive to cancer treatment with a kinase inhibitor, comprising:
providing a nucleic acid sample of the individual;
detecting in the nucleic acid sample the presence or absence of a polymorphic variant encoding the c-kit L576P activating mutation.

14. The method of claim 13, wherein the cancer is melanoma.

15. The method of claim 13, wherein the kinase inhibitor is imatinib.

16. The method of claim 13, wherein the step of detecting the presence or absence of the polymorphic variant encoding the c-kit L576P activating mutation comprises detecting hybridization of an oligonucleotide probe complementary to the nucleic acid encoding the c-kit L576P activating mutation, or a complement thereof.

17. The method of claim 13, wherein the step of detecting the presence or absence of the polymorphic variant encoding the c-kit L576P activating mutation comprises detecting hybridization of an oligonucleotide primer complementary to the nucleic acid encoding the c-kit L576P activating mutation, or a complement thereof.

18. The method of claim 17, wherein the step of detecting the polymorphic variant encoding the c-kit L576P activating mutation includes subjecting said primer to polymerization conditions, such that polymerization of the primer is indicative of the presence in the c-kit polynucleotide of the c-kit L576P activating mutation.

19. The method of claim 13, wherein the individual in need of screening for susceptibility to treatment by a kinase inhibitor has been determined to be resistant to a cancer drug other than a kinase inhibitor.

20. A kit for detecting a single nucleotide polymorphism (SNP) in a nucleic acid, comprising the polynucleotide of claim 6, a buffer, and an enzyme.

21. A kit for identifying a drug susceptibility-conferring mutation, comprising: a suitable container, and at least one of the following: a wild-type c-kit polynucleotide; at least one c-kit polynucleotide comprising a drug resistance-conferring mutation; or a primer that identifies the resistance-conferring mutation.


SEQUENCE LISTING (10 sequences)

SEQ ID NO: 1 - DNA, 240 bp, Human immunodeficiency virus
atgactgagacaataattattaaaaggtgatctatttttccctttctccccacagaaacccatgtatgaagtacagtggaaggttgttgaggagataaatggaaacaattatgtttacatagacccaacacaacytccttatgatcacaaatgggagtttcccagaaacaggctgagttttggtcagtatgaaacaggggctttccatgtcacctttttgggtacacataacagtgactt

SEQ ID NO: 2 - protein, 976 aa, Human immunodeficiency virus [sequence omitted]

SEQ ID NO: 3 - DNA, 22 bp, Human immunodeficiency virus
ctctccagagtgctctaatgac

SEQ ID NO: 4 - DNA, 21 bp, Human immunodeficiency virus
agcccctgtttcatactgacc

SEQ ID NO: 5 - DNA, 20 bp, Human immunodeficiency virus
gatgctctgcttctgtactg

SEQ ID NO: 6 - DNA, 23 bp, Human immunodeficiency virus
gcctaaacatccccttaaattgg

SEQ ID NO: 7 - DNA, 21 bp, Human immunodeficiency virus
cggccatgactgtcgctgtaa

SEQ ID NO: 8 - DNA, 21 bp, Human immunodeficiency virus
ctccaatggtgcaggctccaa

SEQ ID NO: 9 - DNA, 20 bp, Human immunodeficiency virus
tctcctccaacctaatagtg

SEQ ID NO: 10 - DNA, 19 bp, Human immunodeficiency virus
ggactgtcaagcagagaat